(19) United States
(12) Patent Application Publication
(10) Pub. No.: US 2024/0415451 A1
(43) Pub. Date: Dec. 19, 2024

(54) SYSTEM TO MONITOR AND MANAGE PATIENT HYDRATION VIA PLETHYSMOGRAPH VARIABILITY INDEX IN RESPONSE TO THE PASSIVE LEG RAISING

(71) Applicant: Masimo Corporation, Irvine, CA (US)

(72) Inventors: Massi Joe E. Kiani, Laguna Niguel, CA (US); Bilal Muhsin, Irvine, CA (US); Keith Ward Indorf, Lake Elsinore, CA (US)

(21) Appl. No.: 18/661,494

(22) Filed: May 10, 2024

Related U.S. Application Data
(63) Continuation of application No. 16/673,335, filed on Nov. 4, 2019, now Pat. No. 12,004,869.
(60) Provisional application No. 62/755,802, filed on Nov. 5, 2018.

(51) Int. Cl. A61B 5/00; A61B 5/024; A61B 5/026; A61B 5/029; G16H 20/17

(52) U.S. Cl. CPC A61B 5/4848; A61B 5/02405; A61B 5/02433; A61B 5/0261; A61B 5/029; A61B 5/4839; A61B 5/4875; A61B 5/7275; A61B 5/742; G16H 20/17

(57) ABSTRACT

Techniques for predicting fluid responsiveness or fluid unresponsiveness are described. A processor can determine a first prediction of fluid responsiveness or unresponsiveness based on a plethysmograph variability parameter associated with a plethysmograph waveform, and can determine a second prediction of fluid responsiveness or unresponsiveness based on a fluid responsiveness parameter that is associated with an elevation of one or more limbs of the patient. The processor can determine an overall prediction of fluid responsiveness or unresponsiveness based on the first and/or second predictions. Based on overall prediction, the processor can cause administration of fluids and/or termination of administration of fluids.

### RELATED APPLICATIONS

The application is a continuation of U.S. patent application Ser. No. 16/673,335, filed Nov. 4, 2019, which claims priority to U.S. Provisional Application No. 62/755,802, filed Nov. 5, 2018, entitled “System To Manage Patient Hydration,” the entirety of each of which are hereby incorporated by reference in their entirety.

### TECHNICAL FIELD

The present disclosure relates generally to management of patient hydration and assessment of fluids responsiveness.

### BACKGROUND

In the management of patients, the decision to administer fluids constitutes a common dilemma for physicians. For example, although a particular patient may be at least partially dehydrated (for example, due to preoperative fasting, sweat loss, urinary excretion, surgical blood loss, fluid shifts or other pathologic processes), the patient may not respond favorably to fluid administration (e.g., intravenous (IV) therapy). That is, the patient may not respond to fluid administration with an increase in stroke volume.

Accordingly, prior to or during fluid administration, it can be desirable to predict whether the patient's stroke volume (or, in some cases, cardiac output) will increase upon the fluid administration. Unfortunately, accurate prediction of an increase in stroke volume or cardiac output upon fluid loading, so-called fluid responsiveness, has proven to be difficult and/or unreliable.

### SUMMARY

Techniques for predicting fluid responsiveness or fluid unresponsiveness are described. A processor can determine a first prediction of fluid responsiveness or unresponsiveness based on a plethysmograph variability parameter associated with a plethysmograph waveform, and can determine a second prediction of fluid responsiveness or unresponsiveness based on a fluid responsiveness parameter that is associated with an elevation of one or more limbs of the patient. The processor can determine an overall prediction of fluid responsiveness or unresponsiveness based on the first and/or second predictions. Based on overall prediction, the processor can cause administration of fluids and/or termination of administration of fluids.

In various embodiments, the system can identify a patient's hydration status based on physiological data associated with a patient. For example, in some cases, physiological data can correspond to a plethysmography (pleth) signal detected by a pulse oximetry device. Based on the physiological data, the system can identify a pleth variability parameter that is indicative of the hydration status of the patient, and can determine the hydration status of the patient based on the pleth variability parameter. As a non-limiting example, the pleth variability parameter can include a pleth variability index (PVI). For example, PVI can be obtained noninvasively, automatically, and continuously, and can quantify respiratory induced variations in a perfusion index. PVI can be an indicator of a patient's hydration status (for example, hydrated or dehydrated) and can be presented as a numerical value. In some cases, based on PVI the system can determine a hydration status of the patient.

In some cases, a method of determining fluid responsiveness of a patient includes receiving a sensor signal corresponding to a non-invasive physiological sensor. The non-invasive physiological sensor can be configured to emit light towards a tissue site of a patient, detect the light after it has interacted with the tissue site, and generate the sensor signal based at least in part on the detected light. The method can further include demodulating the sensor signal to generate a plethysmograph waveform including a plurality of pulses corresponding to pulsatile blood flow within the tissue site. The method can further include determining a plethysmograph variability parameter associated with the plethysmograph waveform. The plethysmograph variability parameter can quantify variations in the plethysmograph waveform. The method can further include determining a first prediction of fluid responsiveness based at least in part on the plethysmograph variability parameter. The method can further include determining a second prediction of fluid responsiveness based at least in part on a fluid responsiveness parameter that is associated with an elevation of one or more limbs of the patient. The method can further include outputting an indication of fluid responsiveness of the patient based at least in part on the first prediction of fluid responsiveness and the second prediction of fluid responsiveness.

The method of any of the preceding paragraphs and/or any of the methods disclosed herein may include any combination of the following steps or features described in this paragraph, among other features described herein. The plethysmograph variability parameter can correspond to a value associated with a pleth variability index (PVI). The fluid responsiveness parameter can include a measure of at least one of cardiac output, heart rate, or stroke volume. The method can further include determining a plurality of perfusion parameters based at least in part on the plethysmograph waveform. A particular perfusion parameter of the plurality of perfusion parameters can be determined based at least in part on a peak amplitude and a valley amplitude of a corresponding pulse of the plurality of pulses. Said determining the plethysmograph variability parameter can be based at least in part on a difference between a first and second perfusion parameter of the plurality of perfusion parameters relative to the first perfusion parameter of the plurality of perfusion parameters. The indication of fluid responsiveness of the patient can be an overall prediction of fluid responsiveness of the patient.

The method of any of the preceding paragraphs and/or any of the methods disclosed herein may include any combination of the following steps or features described in this paragraph, among other steps or features described herein. The method can be performed prior to administration fluids to the patient. The method can be performed during administration fluids to the patient. The elevation of the one or more limbs of the patient can be associated with a passive leg raising (PLR) test. The PLR test can be performed prior to administration of fluids to the patient. The PLR test can be performed during administration of fluids to the patient. The method can include performing an action based at least in part on the indication of the fluid responsiveness. The action can include at one of initiating administration of fluids to the patient, terminating the administration of the fluids to the patient, causing a display to display an indication of a recommendation for fluid administration, or causing the display to display an indication of a recommendation for termination of fluid administration. The indication of fluid responsiveness can indicate a response of stroke volume of the patient to fluid administration. The method can include determining a first confidence parameter corresponding to the first prediction of fluid responsiveness; and determining a second confidence parameter corresponding to the second prediction of fluid responsiveness. The fluid responsiveness of the patient can be further based at least in part on the first confidence parameter and the second confidence parameter.

In some cases, a system for determining fluid responsiveness of a patient can include a sensor interface and a processor in communication with the sensor interface. The sensor interface can be configured to connect to a non-invasive physiological sensor and to receive a sensor signal from the non-invasive physiological sensor. The non-invasive physiological sensor can be configured to emit light towards a tissue site of a patient, detect the light after it has interacted with the tissue site, and generate the sensor signal based at least in part on the detected light. The processor can be configured to determine a plethysmograph variability parameter associated with a plethysmograph waveform corresponding to the sensor signal. The plethysmograph waveform can include a plurality of pulses corresponding to pulsatile blood flow within the tissue site. The plethysmograph variability parameter can quantify variations in the plethysmograph waveform. The processor can be further configured to determine a first prediction of fluid responsiveness based at least in part on the plethysmograph variability parameter. The processor can be further configured to determine a second prediction of fluid responsiveness based at least in part on a fluid responsiveness parameter that is associated with an elevation of one or more limbs of the patient. The processor can be further configured to output an indication of fluid responsiveness of the patient based at least in part on the first prediction of fluid responsiveness and the second prediction of fluid responsiveness.

The patient monitoring device of any of the preceding paragraphs and/or any of the patient monitoring devices disclosed herein may include any combination of the following features described in this paragraph, among other features described herein. The plethysmograph variability parameter can correspond to a value associated with a pleth variability index (PVI). The fluid responsiveness parameter can include a measure of at least one of cardiac output, heart rate, or stroke volume. The elevation of the one or more limbs of the patient can be associated with a passive leg raising (PLR) test. The PLR test can be performed prior to administration of fluids to the patient. The PLR test can be performed during administration of fluids to the patient. The processor can be further configured to cause an action based on the fluid responsiveness of the patient, wherein the action can include at least one of initiating administration of fluids to the patient, terminating the administration of the fluids to the patient, causing a display to display an indication of a recommendation for fluid administration, or causing the display to display an indication of a recommendation for termination of fluid administration. The processor can be further configured to determine a first confidence parameter corresponding to the first prediction of fluid responsiveness; and determine a second confidence parameter corresponding to the second prediction of fluid responsiveness. The fluid responsiveness of the patient can be further based at least in part on the first confidence parameter and the second confidence parameter.

Any of the features of any of the methods described herein can be used with any of the features of any of the other methods described herein. Any of the features of any of the systems, devices, or methods illustrated in the figures or described herein can be used with any of the features of any of the other systems, devices, or methods illustrated in the figures or described herein.

### BRIEF DESCRIPTION OF THE DRAWINGS

The following drawings and the associated descriptions are provided to illustrate embodiments of the present disclosure and do not limit the scope of the claims.

FIG. 1 is an example block diagram of a patient monitoring device.

FIG. 2A is a perspective view of an example patient monitoring device including a hub and the patient monitoring device of FIG. 1.

FIG. 2B illustrates an example hardware block diagram of the hub of FIG. 2B.

FIG. 3 illustrates a perspective view of the back of the patient monitoring device of FIG. 2A.

FIG. 4 illustrates a pleth signal plotted on an intensity axis versus a time axis.

FIG. 5 is a flow diagram illustrative of an embodiment of a routine for assessing or predicting fluid responsiveness.

FIGS. 6A-6C illustrate embodiments of example GUIs that displays example graphics for instructing or implementing the PLR test on a patient.

FIG. 7 illustrates an embodiment of an example GUI that displays a fluid administration recommendation based on the hydration status and/or fluid responsiveness of the patient.

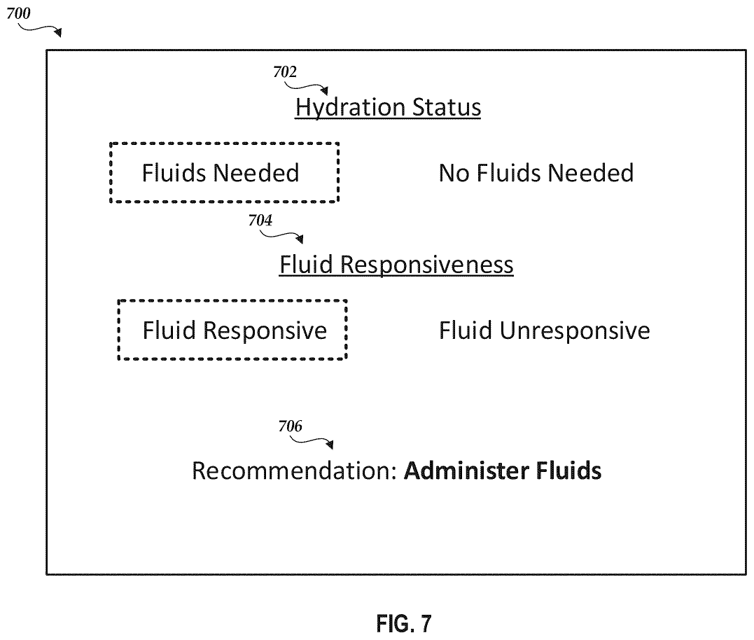

While the foregoing “Brief Description of the Drawings” references generally various embodiments of the disclosure, an artisan will recognize from the disclosure herein that such embodiments are not mutually exclusive. Rather, the artisan would recognize a myriad of combinations of some or all of such embodiments.

### DETAILED DESCRIPTION

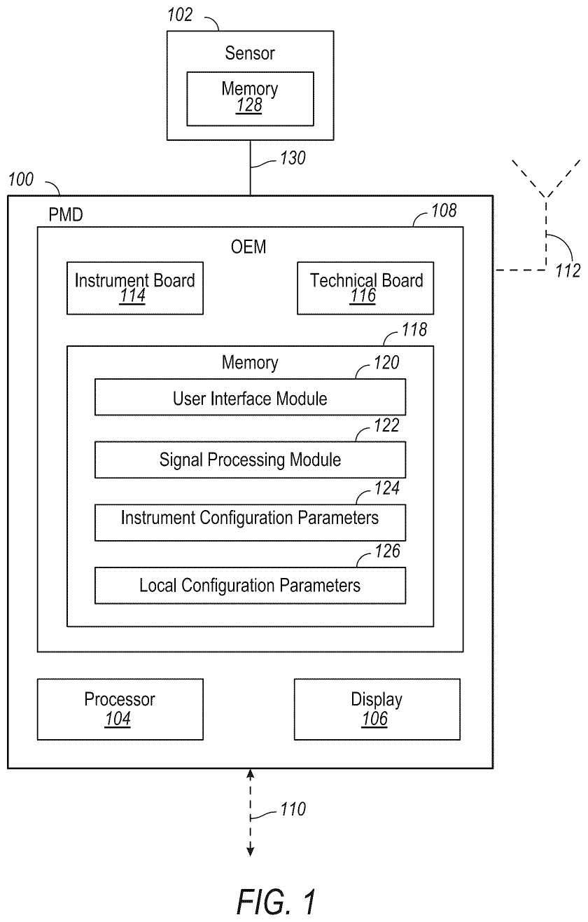

FIG. 1 is an example block diagram of a patient monitoring device 100. The patient monitoring device can include a docked portable patient monitor 100, which may be referred to herein as the patient monitoring device 100. The patient monitoring device 100 may advantageously include an oximeter, co-oximeter, respiratory monitor, depth of sedation monitor, noninvasive blood pressure monitor, vital signs monitor or the like, such as those commercially available from Masimo Corporation of Irvine, CA, and/or disclosed in U.S. Patent Publication Nos. 2002/0140675, 2010/0274099, 2011/0213273, 2012/0226117, 2010/0030040; U.S. Patent Application Ser. Nos. 61/242,792, 61/387,457, 61/645,570, Ser. No. 13/554,908 and U.S. Pat. Nos. 6,157,850, 6,334,065, and the like.

The patient monitoring device 100 can include a first processor 104, a display 106, and an OEM board 108. The patient monitoring device 100 further includes one or more cables 110 and an antenna 112 for wired and wireless communication, respectively.

The OEM board 108 can include an instrument board 114, a core or technical board 116, and memory 118. In some cases, the memory 118 can include a user interface module 120, a signal processing module 122, instrument configuration parameters 124, and local configuration parameters 126.

The patient monitoring device 100 may communicate with a variety of noninvasive and/or minimally invasive sensors 102 such as optical sensors with light emission and detection circuitry, acoustic sensors, devices that measure blood parameters from a finger prick, cuffs, ventilators, ECG sensors, pulse oximeters, and the like.

One or more of the sensors 102 can be attached to a medical patient. The sensors 102 can obtain physiological information from a medical patient and transmit this information to the technical board 116 through cables 130 or through a wireless connection (not shown). The physiological information can include one or more physiological parameters or values and waveforms corresponding to the physiological parameters.

The technical board 116 can receive physiological information from the sensors 102. The technical board 116 can include a circuit having a second processor, which may be the same as the first processor 104, and input ports for receiving the physiological information. The technical board 116 can access the signal processing module 122 to process the physiological information in the second processor. In addition, the technical board 116 can include one or more output ports, such as serial ports. For example, an RS232, RS423, or autobaud RS232 (serial interface standard) port or a universal serial bus (USB) port may be included in the technical board 116.

The technical board 116 and the signal processing module 122 can include a sensor processing system for the patient monitoring device 100. In some cases, the sensor processing system generates waveforms from signals received from the sensors 102. The sensor processing system may also analyze single or multiparameter trends to provide early warning alerts to clinicians prior to an alarm event. In addition, the sensor processing system can generate alarms in response to physiological parameters exceeding certain safe thresholds.

Example alerts include no communication with the patient monitoring device 100, alarm silenced on the patient monitoring device 100, instrument low battery (patient monitoring device 100), transmitter low battery, and/or indications of fluid responsiveness. Example physiological parameters include SpO₂ levels, high and low SpO₂, high and low PR, HbCO level, HbMET level, pulse rate, perfusion index, signal quality, HbCO, HbMET, PI, and desat index. Additional example alarms include SpO₂ alarms, high and low SpO₂ alarms, high and low PR, HbCO alarms, HbMET alarms, pulse rate alarms, no sensor alarms, sensor off patient alarms, sensor error, low perfusion index alarm, low signal quality alarm, HbCO alarm, HbMET alarm, PI trend alarm, and desat index alarm.

The instrument board 114 can receive the waveforms, alerts, alarms, and the like from the technical board 116. The instrument board 114 can include a circuit having a third processor, which may be the same as the first processor 104, and input ports for receiving the waveforms, alerts, and alarms from the technical board 116 and output ports for interfacing with the display 106, a speaker or other device capable of producing an audible indication. The instrument board 114 can access the user interface module 120 to process the waveforms, alerts, and alarms to provide indications of the waveforms, alerts, alarms or other data associated with the physiological parameters monitored by the sensors 102. The indications can be displayed on the display 106. In addition or alternatively, the alerts and alarms are audible. The indications, alerts, and alarms can be communicated to end-user devices, for example, through a hospital backbone, a hospital WLAN 16, and/or the Internet.

Additionally, the instrument board 114 and/or the technical board 116 may advantageously include one or more processors and controllers, busses, all manner of communication connectivity and electronics, memory, memory readers including EPROM readers, and other electronics recognizable to an artisan from the disclosure herein. Each board can include substrates for positioning and support, interconnect for communications, electronic components including controllers, logic devices, hardware/software combinations and the like to accomplish the tasks designated above and others.

An artisan will recognize from the disclosure herein that the instrument board 114 and/or the technical board 116 may include a large number of electronic components organized in a large number of ways.

Because of the versatility needed to process many different physiological parameters, the technical board 116 can further include a revision number or other indication of the circuit design and capabilities of a specific technical board 116.

Likewise, because of the versatility needed to display the processed physiological parameters for use by many different end users, the instrument board 114 can further include a revision number or other indication of the circuit design and capabilities of the specific instrument board.

Software is also subject to upgrading to increase its capabilities. The signal processing module 122 can further include a version number or other indication of the code found in the specific signal processing module 122. Likewise, the user interface module 120 can further include a version number or other indication of the code found on the specific user interface module 120.

Some or all of the serial numbers, the model numbers, and the revision numbers of the technical board 116 and the instrument board 114 that include the specific patient monitoring device 100 can be stored in the instrument configuration parameters 124. Further, the version numbers of the signal processing module 122 and the user interface module 120 are stored in the instrument configuration parameters 124. The instrument configuration parameters 124 can further include indications of the physiological parameters that are enabled, and indications of the physiological parameters that are capable of being enabled for the patient monitoring device 100.

The location of the patient monitoring device 100 can affect the sensitivity at which a physiological parameter is monitored. For example, a physiological parameter may be monitored with greater sensitivity when the patent monitoring device 100 is located in the neonatal intensive care unit (NICU), OR or surgical ICU than when it is located in an adult patient's room. In some cases, the location of the patient monitoring device 100 may affect the availability of the device for another patient. For example, a patient monitoring device 100 located in the hospital discharge area may be available for another patient, whereas one located in a patient's room may not be available anytime soon.

The local configuration parameters 126 can include a location of the patient monitoring device 100 within the facility, an indication of whether the device is configured for adult or pediatric monitoring, and the like.

The sensor 102 can include memory 128. The memory 128 can include information associated with the sensor 102, such as, but not limited to a sensor type, a sensor model number, a sensor revision number, a sensor serial number, and the like.

The patient monitoring device 100 can include a Radical-7® Rainbow SET Pulse Oximeter by Masimo Corporation, Irvine, CA. The OEM board 108 can be produced by Masimo Corporation, Irvine, CA and used by others to produce patient monitoring devices.

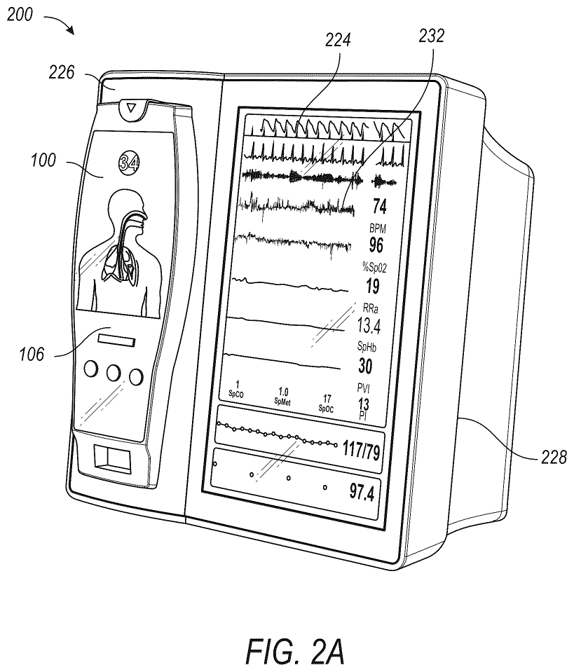

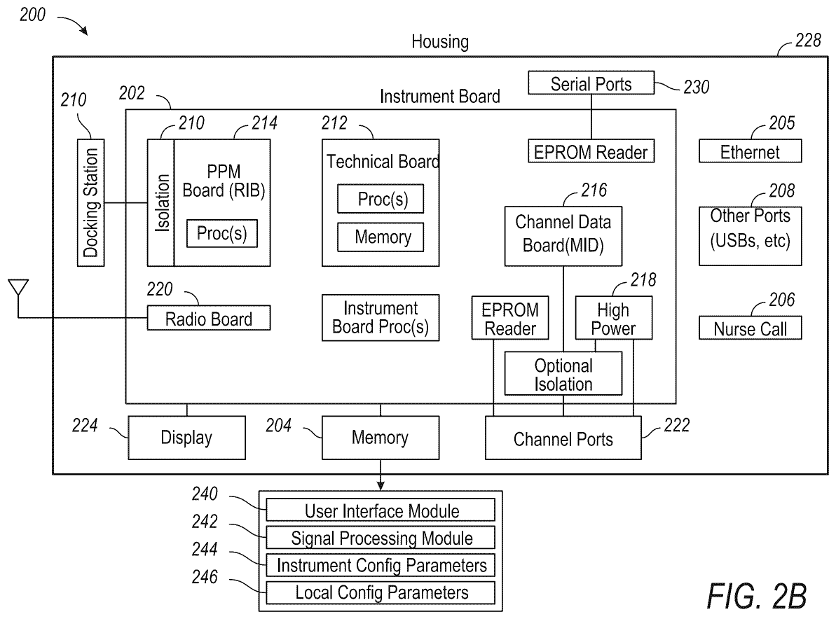

FIG. 2A illustrates a perspective view of an example patient monitoring device, such as a medical monitoring hub with the docked portable patient monitoring device 100, the combination of which may also be referred to herein as a patient monitoring device or patient monitoring system 200. The hub includes a display 224, and a docking station 226, which can be configured to mechanically and electrically mate with the portable patient monitoring device 100, each housed in a movable, mountable and portable housing 228. The housing 228 includes a generally upright inclined shape configured to rest on a horizontal flat surface, although the housing 228 can be affixed in a wide variety of positions and mountings and include a wide variety of shapes and sizes.

The display 224 may present a wide variety of measurement and/or treatment data in numerical, graphical, waveform, or other display indicia 232. The display 224 can occupy much of a front face of the housing 228; although an artisan will appreciate the display 224 may include a tablet or tabletop horizontal configuration, a laptop-like configuration or the like. Other examples may include communicating display information and data to a table computer, smartphone, television, or any display system recognizable to an artisan. The upright inclined configuration of FIG. 2A presents display information to a caregiver in an easily viewable manner. The patient monitoring device 200 may display information for a variety of physiological parameters, such as but not limited to oxygen saturation (SpO2), hemoglobin (Hb), oxyhemoglobin (HbO2), total hemoglobin, carboxyhemoglobin, methemoglobin, perfusion index (Pi), pulse rate (PR) of blood pressure, temperature, electrocardiogram (ECG), motion data, accelerometer data, respiration, continuous blood pressure, pleth variability index (PVI), oxygen content, oxygen reserve index, acoustic respiration rate (RRa), respiration rate from the pleth, cardiac output, stroke volume, and/or fluid responsiveness.

FIG. 2B illustrates a simplified example hardware block diagram of the patient monitoring device 200. As shown in FIG. 2B, the housing 228 of the patient monitoring device 200 positions and/or encompasses an instrument board 202, a core or technical board 212, the display 224, memory 204, and the various communication connections, including serial ports 230, channel ports 222, Ethernet ports 205, nurse call port 206, other communication ports 208 including standard USB, or the like, and a docking station interface 210. The instrument board 202 can include one or more substrates including communication interconnects, wiring, ports and the like to enable the communications and functions described herein, including inter-board communications. The technical board 212 includes the main parameter, signal, and other processor(s) and memory. A portable monitor board (“RIB”) 214 includes patient electrical isolation for the monitor 100 and one or more processors. A channel board (“MID”) 216 controls the communication with the channel ports 222 including optional patient electrical isolation and power supply 218, and a radio board 220 includes components configured for wireless communications.

Additionally, the instrument board 202 and/or the technical board 212 may advantageously include one or more processors and controllers, busses, all manner of communication connectivity and electronics, memory, memory readers including EPROM readers, and other electronics recognizable to an artisan from the disclosure herein. Each board can include substrates for positioning and support, interconnect for communications, electronic components including controllers, logic devices, hardware/software combinations and the like to accomplish the tasks designated above and others.

An artisan will recognize from the disclosure herein that the instrument board 202 and or the technical board 212 may include a large number of electronic components organized in a large number of ways.

Because of the versatility needed to process many different physiological parameters, the technical board 212 can further include a revision number or other indication of the circuit design and capabilities of a specific technical board 212.

Likewise, because of the versatility needed to display the processed physiological parameters for use by many different end users, the instrument board 202 can further include a revision number or other indication of the circuit design and capabilities of the specific instrument board 202.

The memory 204 can include a user interface module 240, a signal processing module 242, instrument configuration parameters 244, and local configuration parameters 246.

The instrument board 202 can access the user interface module 240 to process the waveforms, alerts, and alarms to provide indications of the waveforms, alerts, alarms or other data associated with the physiological parameters for the patient monitoring device 200. The technical board 212 can access the signal processing module 242 to process the physiological information for the patient monitoring device 200.

Software for the patient monitoring device 200 is also subject to upgrading to increase its capabilities. The signal processing module 242 can further include a version number or other indication of the code found in the specific signal processing module 242. Likewise, the user interface module 240 can further include a version number or other indication of the code found on the specific user interface module 240.

Some or all of the serial numbers, the model numbers, and the revision numbers of the technical board 212 and the instrument board 202 that include the specific patient medical monitoring hub 150 can be stored in the instrument configuration parameters 244. Further, the version numbers of the signal processing module 242 and the user interface module 240 can be stored in the instrument configuration parameters 244. The instrument configuration parameters 244 further include indications of the physiological parameters that are enabled, and indications of the physiological parameters that are capable of being enabled for the patient monitoring device 200.

The local configuration parameters 246 can include a location of the patient monitoring device 200 within the facility, an indication of whether the device is configured for adult or pediatric monitoring, and the like.

The patient monitoring device 200 can include a Root® Patient Monitoring and Connectivity Platform by Masimo Corporation, Irvine, CA that includes the Radical-7® also by Masimo Corporation, Irvine, CA.

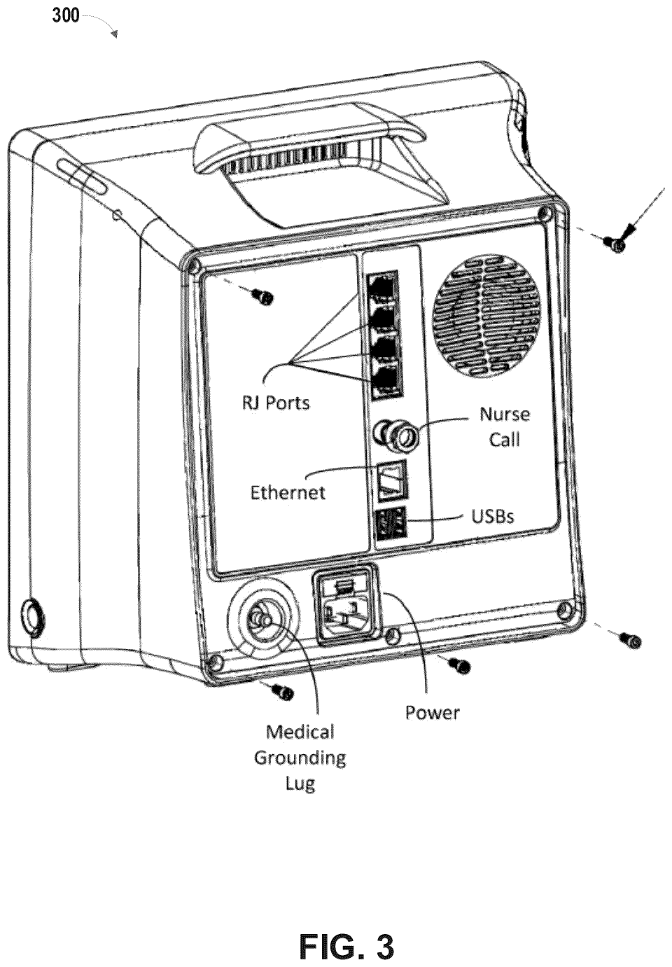

FIG. 3 illustrates an example perspective view of the back of the patient monitoring device 200 of FIG. 2A, showing an example serial data inputs. The inputs can include RJ 45 ports. As is understood in the art, these ports include data ports similar to those found on computers, network routers, switches and hubs. A plurality of these ports can be used to associate data from various devices with the specific patient identified in the patient monitoring device 200. FIG. 3 also shows a speaker, the nurse call connector, the Ethernet connector, the USBs, a power connector and a medical grounding lug.

### Patient Hydration

Dehydration is a condition that can occur when the loss of body fluids, mostly water, exceeds the amount that is taken in. For example, the body is constantly losing fluids through breathing, sweat loss, and urinary excretion. In addition, the body can lose fluids as a result of preoperative fasting, surgical blood loss, fluid shifts or other pathologic processes. With dehydration, more water is moving out of individual cells and then out of the body than the amount of water that is taken in. Medically, dehydration usually means a person has lost enough fluid so that the body begins to lose its ability to function normally.

The severity of dehydration can vary based on the amount of fluids in the patient's body. For example, in some cases, dehydration can be classified based on levels or a degree of dehydration. For instance, in some cases, if a patient is dehydrated, a level of the patient's dehydration can be classified as one of mild dehydration, moderate dehydration, or severe dehydration, or somewhere in between. In some cases, mild dehydration corresponds to a 3% to 5% drop in fluids as compared to normal or average fluid levels of the patient, moderate dehydration corresponds to a 6% to 10% drop in fluids as compared to normal or average fluid levels of the patient, and severe dehydration corresponds to more than a 10% drop in fluids as compared to normal or average fluid levels of the patient. However, it will be understood that the range or categorizations of degrees of dehydration can vary across embodiments. For example, in some cases, patient hydration can be binary: dehydrated or not dehydrated. Furthermore, in some cases, patient hydration can correspond to a sliding scale, such as a scale from 0 to 100. Further still, the ranges or categorizations of degrees of dehydration can vary based on a patient's age, gender, weight, etc.

Dehydration can itself be dangerous. Furthermore, dehydration can cause common conditions including, but not limited to, constipation, falls, urinary tract infections, pressure ulcers, malnutrition, incontinence, confusion, acute kidney injury, cardiac disease or venous thromboembolism. For some patients, preventing or treating dehydration is difficult without assistance. For instance, a patient may not be able to drink or consume fluids. In some such cases, fluids can be administered to the patient, for example, intravenously. However, in many instances, dehydrated can be difficult to detect or predict. Accordingly, it can be desirable to gage an accurate assessment of patient hydration or dehydration.

In some cases, the system can obtain an accurate assessment of patient hydration or dehydration (non-limiting example, whether a hydration threshold is satisfied or not satisfied) using physiological data of the patient. For example, the system can include (or receive signals from) a pulse oximeter, which can be positioned on the patient. In some cases, the pulse oximeter can detect a pleth signal, and can communicate the pleth signal, or an indication thereof, to the system.

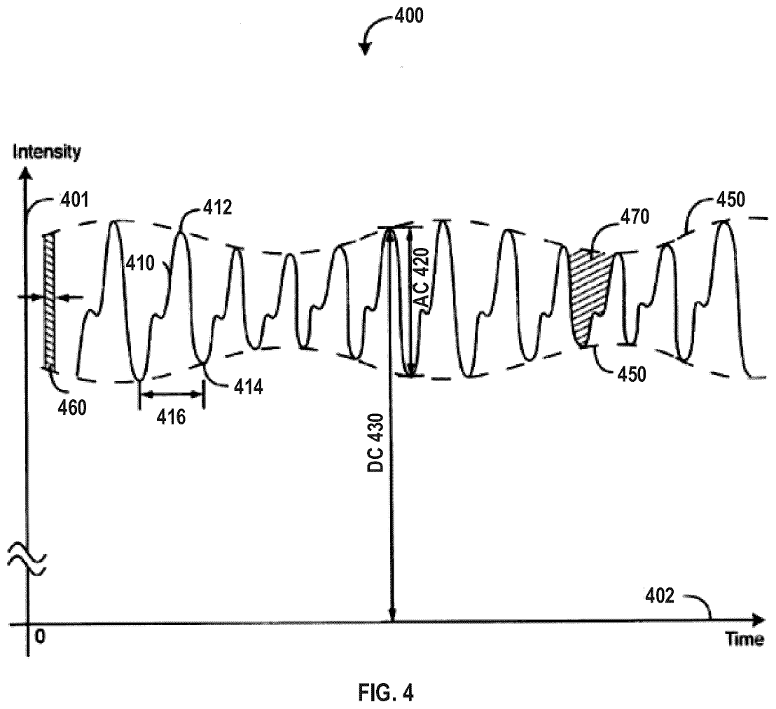

FIG. 4 illustrates a pleth signal 400 plotted on an intensity axis 401 versus a time axis 402. The pleth signal 400 has multiple pulses 410, which correspond to pulsatile blood flowing within a tissue site. As illustrated, each pulse 410 is characterized by a plurality of features such as a peak amplitude 412, valley amplitude 414, and period 416. Further, pleth signal 400 defines a pleth envelope 450 interpolated from pulse peaks 412 and pulse valleys 414.

Perfusion values, or a perfusion index (PI), can be defined for each pulse 410. PI generally reflects the amplitude of the waveform and is calculated as the pulsatile infrared signal (AC 420 or variable component), indexed against the non-pulsatile infrared signal (DC 430 or constant component). PI can be expressed as a percentage (for example, 0.02-20%). In some cases, PI is the ratio of the pulsing blood to non-pulsing blood flow, and can be determined using the following equation:

$$P I = \frac{A C}{D C} \qquad ( Equation 1 )$$

where AC can represent a peak amplitude 412 minus a valley amplitude 414 for a particular pulse, and DC can represent a peak amplitude 412 for a particular pulse. Among other things, PI can be used to indicate a strength of blood flow a measurement site. In some cases, DC can represent a value other than peak amplitude 412, such as a valley amplitude 414 or an average of peak amplitude 412 and valley amplitude 414, to name a few.

In some cases, a patient monitoring device, such as the patient monitoring device 100 of FIG. 1, can identify a pleth variability parameter that is responsive to variations of the pleth. In some cases, a pleth variability parameter is a clinically useful hemodynamic measurement because it can respond to changes in patient physiology, thereby acting as a useful indicator of various physiological conditions or the efficacy of treatment for those conditions. Advantageously, pleth variability measures may provide a numerical indication of a person's physical condition or health. For example, changes in a pleth variability parameter may be representative of changes in physiologic factors such as patient hydration.

One variability measure is a Pleth Variability Index (PVI or PVi®) (developed by Masimo® Corporation, Irvine, CA, USA)) as described in greater detail in U.S. Pub. No. 2008/0188760, filed Dec. 7, 2007, and entitled “PLETHYSMOGRAPH VARIABILITY PROCESSOR,” which is hereby incorporated by reference herein in its entirety.

PVI can be based on perfusion index (PI) variations during a respiratory cycle. For example, PVI can be a measure of dynamic changes in PI that occur during one or more complete respiratory cycles. As illustrated from Equation 2 below, the calculation for PVI can be accomplished by measuring changes in PI over a time interval where one or more complete respiratory cycles have occurred, and can be determined using the following equation:

$$P V I = \frac{{PI}_{max} - {PI}_{min}}{{PI}_{max}} * 100 \qquad ( Equation 2 )$$

In some cases, PVI can be automatically or continuously calculated and can represent respiratory variations in the plethysmographic waveform. Furthermore, in some cases, PVI may be indicative of or correlated with patient hydration or dehydration. As a non-limiting example, in some cases, a relatively lower PVI can indicate that the patient's hydration level does not satisfy a hydration threshold (e.g., the patient is dehydrated). As a corollary, a relatively high PVI can indicate that the patient's hydration level satisfies a hydration threshold (e.g., the patient is not dehydrated). However, it will be understood that, in some cases, a relatively low PVI can indicate that the patient's hydration level satisfies a hydration threshold and/or a relatively high PVI can indicate that the patient's hydration level does not satisfy a hydration threshold. In some cases, PVI can be displayed as a percentage (numerical value) and/or a trend graph. Similarly, the patient monitoring device can display (or cause a display to display) an indication of the patient's hydration status (for example, dehydrated, hydrated, no dehydrated, mildly dehydration, moderately dehydration, or severely dehydration mild dehydration, etc.) based at least in part on PVI. As another example, in some cases, the patient monitoring device can provide an indication to (or control a device to) initiate administration of fluids, continue administration of fluids, or terminate administration of fluids based at least in part on PVI. For example, in some cases, based on a determination that the patient is dehydrated, the patient monitoring device can provide an indication to (or control a device to) initiate administration of fluids or continue administration of fluids. As a corollary, in some cases, based on a determination that the patient is not dehydrated, the patient monitoring device can provide an indication to (or control a device to) terminate administration of fluids.

### Cardiac Output

Cardiac output can refer to an amount or volume of blood that the heart pumps through the circulatory system, and is generally expressed in liters per minute. Sufficient cardiac output (that is, a cardiac output that satisfies a threshold cardiac output) helps keep blood pressure at the levels needed to supply oxygen-rich blood to the brain and other vital organs. In some cases, cardiac output can be calculated using the following equation:

$$C O = H R \times S V \qquad ( Equation 3 )$$

where CO is the Cardiac output, HR is heart rate (e.g., the number of heart beats per minute (bpm)), and SV is stroke volume (e.g., the amount of blood pumped from the left (or right) ventricle of the heart in one contraction). Accordingly, the cardiac output can be a function of heart rate and stroke volume, and thus, in some cases, the factors affecting stroke volume or heart rate may also affect cardiac output.

As a non-limiting example, for someone weighing about 70 kg (154 lbs.), a healthy heart with a normal cardiac output can pump about 5 to 6 liters of blood every minute when a person is resting. During exercise, the body may need three or four times its normal cardiac output, because the muscles need more oxygen. Thus, during exercise, the heart typically beats faster (known as increased heart rate) so that more blood flows out to the body. The heart can also increase its stroke volume by pumping more forcefully or increasing the amount of blood that fills the left ventricle before it pumps. Generally, an increase in cardiac output can be favorable or desired, as it can indicate an increase in the supply of oxygen-rich blood. In contrast, a decrease in cardiac output can be unfavorable or not desired, as it can indicate a decrease in the supply of oxygen-rich blood.

### Fluid Responsiveness

Volume expansion can be applied to increase an amount of fluid present in a patient's body, and is frequently used before, during, or after surgery to correct dehydration or fluid deficits created by, for example, preoperative fasting, surgical blood loss, sweat loss, urinary excretion, fluid shifts or other pathologic processes. Techniques for volume expansion include, among other methods, oral rehydration therapy (for example, drinking), intravenous therapy (for example, delivering liquid substances directly into a vein), rectal therapy (for example, with a Murphy drip), or by hypodermoclysis (for example, the direct injection of fluid into the subcutaneous tissue).

In general, the objective of volume expansion is to improve oxygen delivery or overall hemodynamic function. However, some patients may not respond to volume expansion with improved oxygen delivery or improved overall hemodynamic function. For example, these patients may not respond to the volume expansion with an increase in stroke volume or cardiac output. In these patients, volume expansion can be either ineffective or deleterious, and can result in worsening oxygen delivery, inducing systemic and pulmonary edema or, in some cases, cardiac failure. In some cases, patients that do not respond to volume expansion with improved oxygen delivery or improved overall hemodynamic function are referred to as being fluid unresponsive. In some cases, patients that do respond to volume expansion with improved oxygen delivery or improved overall hemodynamic function are referred to as being fluid responsive. Thus, in some cases, a patient can be categorized as fluid unresponsive or fluid responsive based on how he or she will respond to volume expansion (sometimes referred to as a patient's fluid responsiveness).

As described herein, some patient's may be fluid unresponsive. Thus, it can be important to determine or predict a patient's fluid responsiveness (e.g. how a patient will respond to volume expansion) prior to administering fluids. In other words, prior to fluid administration, it can be important to determine or predict a patient's fluid responsiveness (or how fluid administration will affect a patient's cardiac output). A patient identified as someone that will respond to volume expansion with an increase in cardiac output such that the cardiac output satisfies a threshold cardiac output (or that the increase in cardiac output satisfies a threshold increase in cardiac output) can be classified as fluid responsive. In contrast, a patient identified as someone that will not respond to volume expansion with a cardiac output that satisfies a threshold cardiac output can be classified as fluid unresponsive. In some cases, an indication of a patient's fluid responsiveness can include an indication that the patient is fluid responsive, fluid unresponsive, or somewhere in between.

Although some static and dynamic cardiopulmonary indices have been used to predict fluid responsiveness, many of these measures generally have low predictive value, or can risk fluid overload (the condition of having too much fluid in the body, or the state of one of the chambers of the heart in which too large a volume of blood exists within it for it to function efficiently). For example, a fluid challenge can include administering fluids to patients in order to assess their response to fluid therapy and guide further treatment decisions. By administering a small amount of fluid in a short period of time, the clinician can assess the patient's fluid responsiveness. However, because volume expansion includes administering fluid to the patient, a fluid challenge can risk fluid overload.

### Passive Leg Raising (PLR) Test

In some cases, the system can utilize information obtained as a result of the patient performing or being administered a passive leg raising (PLR) test to determine the patient's fluid responsiveness. The PLR test can vary across embodiments, but is generally a non-invasive, bedside test that involves elevating a patient's legs, and can be used to evaluate whether a patient will benefit from volume expansion. In some cases, the PLR test can be used to determine whether cardiac output respond such that it satisfies or does not satisfy a cardiac output threshold as a result of volume expansion.

In general, the PLR test involves raising the legs of a patient (without her active participation), which causes gravity to pull blood from the legs, thus increasing circulatory volume available to the heart, sometimes known as cardiac preload. The PLR test can be performed with the patient's active participation. For instance, the patient can actively raise his or legs. The PLR test can be performed with the patient's active participation. By transferring a volume of blood (for example, around 300 mL) from the lower body toward the heart, PLR mimics a fluid challenge. However, no fluid is infused and the hemodynamic effects are rapidly reversible, thereby avoiding the risks of fluid overload.

A method for performing a PLR test can include a sequence of steps. For example, the PLR test generally includes some combination of the following steps: (1) placing the patient in a semi-recumbent position (the patient's head and torso are positioned upright at an angle of about 45° relative to the patient's legs, which are resting flat on the table); (2) assessing the cardiac output. Here, the patient's heart rate, stroke volume, or the like can be identified by the system, or cardiac output can be determined; (3) moving the patient to a recumbent position (the patient's legs are raised and torso is lowered, where, ultimately, the patient is lying on her back with her feet raised at an angle of about 45°. Here, it may be beneficial not to have the patients elevate her legs manually because it may provoke pain, discomfort, or awakening that can cause adrenergic stimulation, giving false readings of cardiac output by increasing heart rate); (4) re-assessing the cardiac output. Here, the patient's heart rate, stroke volume, or the like can be identified by the system, or cardiac output can be determined; (5) returning the patient to a semi-recumbent position; and (6) re-assessing the cardiac output. Here, the patient's heart rate, stroke volume, or the like can be identified by the system, or cardiac output can be determined.

In general, the PLR test can be can used to assess fluid responsiveness without any fluid challenge, where the latter can lead to fluid overload. The real-time effects of the PLR test on hemodynamic parameters such as blood pressure, heart rate, or cardiac output can be used to guide the assessments on the patient's fluid responsiveness. For example, in some cases, if the patient's cardiac output responds such that it satisfies a threshold cardiac output, then it can be determined that the patient is fluid responsive. In contrast, in some cases, if the patient's cardiac output responds such that it does not satisfy a threshold cardiac output, then it can be determined that the patient is fluid unresponsive.

### Flow Diagrams

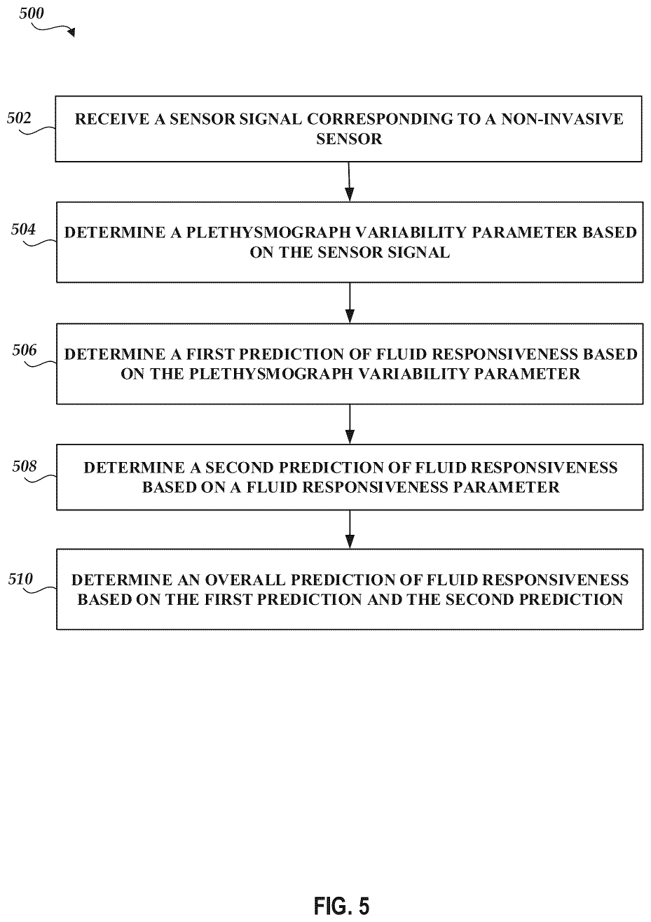

FIG. 5 is a flow diagram illustrative of an embodiment of a routine 500, implemented by a processor, for assessing or predicting fluid responsiveness. One skilled in the relevant art will appreciate that the elements outlined for routine 500 can be implemented by one or more computing devices that are associated with the system 200, such as the processor 104 or the monitoring device 100. Accordingly, routine 500 has been logically associated as being generally performed by the processor 104 of FIG. 1. However, the following illustrative embodiment should not be construed as limiting. Furthermore, it will be understood that the various blocks described herein with reference to FIG. 5 can be implemented in a variety of orders. For example, the processor 104 can implement some blocks concurrently or change the order, as desired. Furthermore, it will be understood that fewer, more, or different blocks can be used as part of the routine 500.

At block 502, the processor 104 receives a sensor signal corresponding to a non-invasive physiological sensor. As described herein, the non-invasive physiological sensor, such as sensor 102 of FIG. 1, can include at least one emitter configured to emit light at one or more wavelengths. Further, the non-invasive sensor can include a detector configured to detect the light from the least one emitter after the light has interacted with the tissue site of a patient, and generate the sensor signal based at least in part on the detected light. For example, the non-invasive physiological sensor can be attached to the patient, such as to the patient's finger.

At block 504, the processor 104 determines a plethysmograph variability parameter based at least in part on the sensor signal. In some cases, the plethysmograph variability parameter can includes a value that relates or quantifies changes in patient physiology. For example, in some cases, the plethysmograph variability parameter can reflect variations that occur during the respiratory cycle or changes in physiologic factors such as changes in fluid responsiveness, volemia, ventricular preload, etc.

The plethysmograph variability parameter can be determined using one or more of various techniques, such as any one or more of the techniques disclosed in U.S. Patent Publication No. 2013/0296713, filed Apr. 8, 2013, which is hereby incorporated by reference in its entirety. For example, the processor 104 can determine perfusion values, or a perfusion index (PI), from pulses of a plethysmograph waveform that corresponds to the sensor signal. Furthermore, the processor 104 can determine the plethysmograph variability parameter based on the perfusion values or the PI. For instance, the determination of the plethysmograph variability parameter can include calculating a difference between perfusion values and normalizing the difference. In some cases, the plethysmograph variability parameter corresponds to one or more plethysmograph variability index (PVI) values. For example, a PVI value can be a measure of the dynamic changes in the Perfusion Index (PI) that occur during one or more complete respiratory cycles.

At block 506, the processor 104 determines a prediction of fluid responsiveness or unresponsiveness based at least in part on the plethysmograph variability parameter. For example, in some cases, the plethysmograph variability parameter includes a numerical value. In some such cases, a relatively higher plethysmograph variability parameter may indicate fluid responsiveness. For example, a higher plethysmograph variability parameter may indicate more variance in the perfusion values and a greater likelihood that the patient will respond to fluid administration with an increase in cardiac output. As a corollary, in some such cases, a relatively lower plethysmograph variability parameter may indicate fluid unresponsiveness. For example, a lower plethysmograph variability parameter may indicate less variance in the perfusion values and therefore a lower likelihood that the patient will respond to fluid administration with an increase in cardiac output.

In some cases, the processor 104 can further determine a confidence value associated with the prediction of fluid responsiveness or unresponsiveness. For example, in some cases, the confidence in a prediction of fluid responsiveness increases (i.e., a higher confidence) as the plethysmograph variability parameter increases and decreases (i.e., a lower confidence) as the plethysmograph variability parameter decreases. As a corollary, in some cases, the confidence in a prediction of fluid unresponsiveness increases as the plethysmograph variability parameter decreases and decreases as the plethysmograph variability parameter increases.

At block 508, the processor 104 determines a prediction of fluid responsiveness or unresponsiveness based at least in part on a fluid responsiveness parameter that is associated with an elevation of one or more limbs of the patient. For example, in some cases, some variance of a passive leg raise (PLR) test can be performed on the patient. As described herein, a PLR test is a non-invasive, bedside test, which can results in the elevation of one or more limbs (e.g., one or more legs) of the patient.

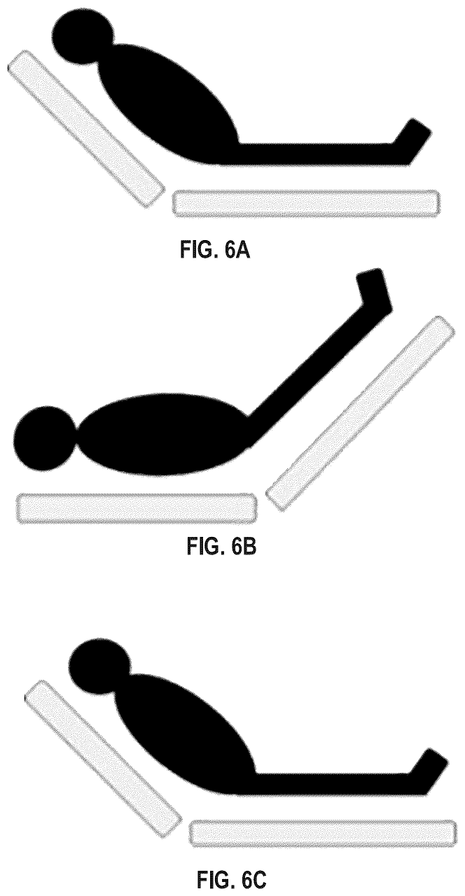

In some cases, the PLR test can be automated or semi-automated. For example, the processor 104 can cause the PLR test to begin, for instance by controlling movement of a patient's bed. In some cases, the PLR test can be performed manually, such as by a physician. In some cases, the processor 104 can provide an instruction to perform the PLR test. The instruction can include an auditory, visual or other indication. In some cases, the processor 104 can provide instructions throughout the PLR test. For example, the processor 104 can generate a graphical user interface (GUI) that displays a graphical indication of one or more steps of the PLR test. In some cases, the processor 104 can cause a display (for example, display 106 or 224) to display a visual walkthrough of the steps of the PLR test. FIGS. 6A-6C illustrate an embodiment of an example GUI that displays example graphics for instructing or implementing the PLR test on a patient. As illustrated, for one or more steps of the PLR test, the GUI can show a graphical depiction of the orientation of the patient and/or the patient's bed. In addition or alternatively, the GUI can include instructions for the caregiver, such as an indication of when to obtain results from the PLR test, a timer or alarm for a particular step, or the like.

The fluid responsiveness parameter can vary across embodiments. For example, in some cases, the fluid responsiveness parameter includes a measure of cardiac output. As another example, in some cases, the fluid responsiveness parameter includes a measure of heart rate. As another example, in some cases, the fluid responsiveness parameter includes a measure of stroke volume. The fluid responsiveness parameter can be determined at one or more of various stages of the PLR test. For example, the fluid responsiveness parameter can be determined prior to performance of the PLR test, during the PLR test, and/or after the PLR test has completed. Furthermore, the fluid responsiveness parameter can be measured in real-time by one or more sensors and/or can be calculated by the processor 104 using sensor data. Alternatively, in some cases, a physician and/or medical device can monitor the fluid responsiveness parameter of the patient, and the physician can enter fluid responsiveness parameter as an input to the processor 104.

The processor 104 can determine a prediction of fluid responsiveness or unresponsiveness in various ways. For example, changes in the fluid responsiveness parameter can be used to guide the determination of the prediction. For example, in some cases, a first fluid responsiveness parameter is measured prior to during the PLR test and a second fluid responsiveness parameter is measured during or after PLR test. In some such cases, the processor 104 can determine a prediction of fluid responsiveness or unresponsiveness based one a comparison for the first fluid responsiveness parameter and the second fluid responsiveness parameter. For example, if the second fluid responsiveness parameter increases (for example, by a threshold amount) relative to the first fluid responsiveness parameter, then the processor 104 can determine a prediction of fluid responsiveness. Put another way, in some cases, if the PLR test causes the fluid responsiveness parameter to increase (for example, by a threshold amount), the processor 104 can determine a prediction of fluid responsiveness. As a corollary, in some cases, if the PLR test causes the fluid responsiveness parameter to stay the same, decrease, or not increase by threshold amount, the processor 104 can determine a prediction of fluid unresponsiveness.

In some cases, the processor 104 can further determine a confidence value associated with the prediction of fluid responsiveness or unresponsiveness. For example, in some cases, the confidence in a prediction of fluid responsiveness or unresponsiveness is based on the amount that the fluid responsiveness parameter changes over time. For example, for a prediction of fluid responsiveness, a relatively larger increase in the fluid responsiveness parameter can result in a higher confidence value, while a relatively smaller increase can result in a lower confidence value. As another example, for a prediction of fluid unresponsiveness, no increase or a decreases in the fluid responsiveness parameter can result in a higher confidence value, while an increase in the fluid responsiveness parameter can result in a lower confidence value.

At block 508, the processor 104 outputs an indication of an overall prediction of fluid responsiveness or fluid responsiveness. For example, the processor 104 can determine the overall prediction of fluid responsiveness or fluid responsiveness based at least in part on the determinations at block 506 and/or block 508. For example, the overall prediction of fluid responsiveness or fluid responsiveness can be based at least in part on one or more of the prediction of fluid responsiveness or unresponsiveness using the plethysmograph variability parameter, the prediction of fluid responsiveness or unresponsiveness using the fluid responsiveness parameter, and/or confidence values associated therewith.

As an example, if both blocks 506 and 508 return a prediction of fluid responsiveness, then the processor 104 can output an indication of fluid responsiveness. As an example, if both blocks 506 and 508 return a prediction of fluid unresponsiveness, then the processor 104 can output an indication of fluid unresponsiveness. In some cases, if one of blocks 506 and 508 returns a prediction of fluid unresponsiveness and one of blocks 506 and 508 returns a prediction of fluid responsiveness, then the processor 104 can output an error code. In some cases, if one of blocks 506 and 508 returns a prediction of fluid unresponsiveness and one of blocks 506 and 508 returns a prediction of fluid responsiveness, then the processor 104 can output the prediction of whichever method produced a higher confidence value, as described herein.

It will be understood that the various blocks described herein can be implemented in a variety of orders, and that the processor 104 can implement one or more of the blocks concurrently and/or change the order, as desired. For example, in some cases, any of blocks 502, 504, 506 and/or 508 can be implemented prior to or currently with any other blocks 502. Furthermore, it will be understood that fewer, more, or different blocks can be used as part of the routine 500. For example, the routine 500 can include blocks for controlling a device to administer fluids to the patient or terminate administration of fluids to the patient. For instance, in some cases, based on a prediction of fluid responsiveness, the processor can cause fluid to be administered to the patient, either by operating or controlling a medical device, such as an infusion pump, to administer fluids to the patient or by outputting an indication to administer fluids. As another example, in some cases, based on a prediction of fluid unresponsiveness, the processor can cause administration of fluid to be terminated, either by operating or controlling a medical device, such as an infusion pump, or by outputting an indication to termination administration of fluids.

Furthermore, the processor 104 can cause display on the GUI of one or more instructions, which, when viewed, can indicate how to perform the PLR test, or a duration over which to hold a specific step of the PLR test. Furthermore, in some cases, rather than or in addition to predicting fluid responsiveness or fluids unresponsiveness, the processor 104 can identify a hydration status of a patient. In some cases, if the patient is hydrated, the processor 104 doesn't initiate the routine 500. In some cases, the processor 104 initiates the routine 500 based on a determination that the patient is dehydrated. Furthermore, in some cases, the routine 500 can omit certain blocks, such as, but not limited to, blocks 502, 504, 506, and/or 508.

### Terminology

The term “and/or” herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

The following description is merely illustrative in nature and is in no way intended to limit the disclosure, its application, or uses. For purposes of clarity, the same reference numbers will be used in the drawings to identify similar elements. It should be understood that steps within a method may be executed in different order without altering the principles of the present disclosure.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figures. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional or alternative components. These components can include menus, lists, buttons, text boxes, labels, radio buttons, scroll bars, sliders, checkboxes, combo boxes, status bars, dialog boxes, windows, and the like. User interface screens can include additional or alternative information. Components can be arranged, grouped, displayed in any suitable order.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. (canceled)

2. A patient monitoring device for determining fluid responsiveness of a patient, the patient monitoring device comprising:
one or more non-invasive physiological sensors configured to obtain physiological information from the patient, and further configured to emit light towards a tissue site of a patient, and detect the light after the light interacted with the tissue site, and generate a sensor signal based at least in part on the detected light;
a sensor interface configured to receive the sensor signal from the one or more non-invasive physiological sensors; and
a processor in communication with the sensor interface and configured to:
automatically determine a plethysmograph variability parameter associated with a plethysmograph waveform corresponding to the sensor signal, wherein the plethysmograph waveform comprises a plurality of pulses corresponding to pulsatile blood flow within the tissue site, wherein the plethysmograph variability parameter quantifies variations in the plethysmograph waveform;
determine an initial hydration status of the patient based at least in part on the plethysmograph variability parameter;
in response to determining the initial hydration status meets a threshold, provide instructions via a graphical user interface (GUI) displayed as a graphical indication of one or more steps of a passive leg raising (PLR) test, wherein the graphical indication of the one or more steps includes example graphics for implementing the PLR test;
determine a first prediction of fluid responsiveness based at least in part on a fluid responsiveness parameter that is associated with a change in elevation of one or more limbs of the patient that is associated with the PLR test;
determine a second prediction of fluid responsiveness based at least in part on the plethysmograph variability parameter;
determine an overall indication of fluid responsiveness of the patient based at least in part on the first prediction and the second prediction of fluid responsiveness; and
based on the fluid responsiveness of the patient, generate a treatment recommendation output providing one or more recommendations regarding administration of fluid to the patient.

3. The patient monitoring device of claim 1, wherein the processor is further configured to provide the instructions via the GUI to display the graphical indication of the one or more steps, depicting an orientation of the patient, of the PLR test.

4. The patient monitoring device of claim 1, wherein the processor is further configured to provide the instructions via the GUI to display the graphical indication of one or more steps, depicting an orientation of a bed of the patient, of the PLR test.

5. The patient monitoring device of claim 1, wherein the processor is further configured to provide the instructions, indicating when to obtain results from performing the PLR test, via the GUI to display the graphical indication of the one or more steps of the PLR test, wherein the instructions comprise one or more of a timer or alarm.

6. The patient monitoring device of claim 1, wherein the processor is further configured to generate an output of a hydration status to display via the GUI.

7. The patient monitoring device of claim 1, wherein the treatment recommendation output comprises one or more of a hydration status, a fluid responsiveness classification, and a fluid administration recommendation.

8. A patient monitoring method for determining fluid responsiveness of a patient, the patient monitoring method comprising:
automatically determining a plethysmograph variability parameter associated with a plethysmograph waveform corresponding to a sensor signal, wherein the plethysmograph waveform comprises a plurality of pulses corresponding to pulsatile blood flow within a tissue site, wherein the plethysmograph variability parameter quantifies variations in the plethysmograph waveform;
determining an initial hydration status of the patient based at least in part on the plethysmograph variability parameter;
in response to determining the initial hydration status meets a threshold, providing instructions via a graphical user interface (GUI) to display a graphical indication of one or more steps of a passive leg raising (PLR) test, wherein the graphical indication of the one or more steps includes example graphics for instructing or implementing the PLR test;
determining a first prediction of fluid responsiveness based at least in part on a change in the fluid responsiveness parameter that is associated with a change in elevation of one or more limbs of the patient that is associated with the PLR test;
determining a second prediction of fluid responsiveness based at least in part on the plethysmograph variability parameter;
determining an overall indication of fluid responsiveness of the patient based at least in part on the first prediction and the second prediction of fluid responsiveness; and
based on the fluid responsiveness of the patient, generating a treatment recommendation output providing one or more recommendations regarding administration of fluid to the patient.

9. The patient monitoring method of claim 7, wherein providing the instructions via the GUI to display the graphical indication of the one or more steps of the PLR further comprises providing the instructions via the GUI to display the graphical indication of the one or more steps of the PLR test depicting an orientation of the patient.

10. The patient monitoring method of claim 7, wherein providing the instructions via the GUI to display the graphical indication of the one or more steps of the PLR further comprises providing the instructions via the GUI to display the graphical indication of one or more steps of the PLR test depicting an orientation of a bed of the patient.

11. The patient monitoring method of claim 7, wherein providing the instructions via the GUI to display the graphical indication of the one or more steps of the PLR further comprises providing the instructions via the GUI to display an indicator of when to obtain results from the PLR test and the graphical indication of the one or more steps of the PLR test, wherein the indicator comprises one or more of a timer or alarm.

12. The patient monitoring method of claim 7, further comprising generating an output of a hydration status to display via the GUI.

13. The patient monitoring method of claim 7, wherein the treatment recommendation output comprises one or more of a hydration status, a fluid responsiveness classification, and a fluid administration recommendation.

14. The patient monitoring method of claim 7, wherein generating the treatment recommendation output comprises generating the treatment recommendation output to display via the GUI.

15. Non-transitory, computer-readable storage media comprising computer-executable instructions for determining fluid responsiveness of a patient, wherein the computer-executable instructions, when executed by a processor, cause the processor to:
automatically determine a plethysmograph variability parameter associated with a plethysmograph waveform corresponding to a sensor signal, wherein the plethysmograph waveform comprises a plurality of pulses corresponding to pulsatile blood flow within a tissue site, wherein the plethysmograph variability parameter quantifies variations in the plethysmograph waveform;
determine an initial hydration status of the patient based at least in part on the plethysmograph variability parameter;
in response to determining the initial hydration status meets a threshold, provide instructions via a graphical user interface (GUI) to display a graphical indication of one or more steps of a passive leg raising (PLR) test, wherein the graphical indication of the one or more steps includes example graphics for instructing or implementing the PLR test;
determine a first prediction of fluid responsiveness based at least in part on a change in a fluid responsiveness parameter that is associated with a change in elevation of one or more limbs of the patient that is associated with the PLR test;
determine a second prediction of fluid responsiveness based at least in part on the plethysmograph variability parameter;
determine an overall indication of fluid responsiveness of the patient based at least in part on the first prediction and the second prediction of fluid responsiveness; and
based on the fluid responsiveness of the patient, generate a treatment recommendation output providing one or more recommendations regarding administration of fluid to the patient.

16. The non-transitory, computer-readable storage media of claim 14, wherein the computer-executable instructions further cause the processor to provide the instructions via the GUI to display the graphical indication of the one or more steps, depicting an orientation of the patient, of the PLR test.

17. The non-transitory, computer-readable storage media of claim 14, wherein the computer-executable instructions further cause the processor to provide the instructions via the GUI to display the graphical indication of one or more steps, depicting an orientation of a bed of the patient, of the PLR test.

18. The non-transitory, computer-readable storage media of claim 14, wherein the computer-executable instructions further cause the processor to provide the instructions, indicating when to obtain results from performing the PLR test, via the GUI to display the graphical indication of the one or more steps of the PLR test, wherein the instructions comprise one or more of a timer or alarm.

19. The non-transitory, computer-readable storage media of claim 14, wherein the computer-executable instructions further cause the processor to generate an output of a hydration status to display via the GUI.

20. The non-transitory, computer-readable storage media of claim 14, wherein the treatment recommendation output comprises one or more of a hydration status, a fluid responsiveness classification, and a fluid administration recommendation.

21. The non-transitory, computer-readable storage media of claim 14, wherein the computer-executable instructions further cause the processor to generate the treatment recommendation output to display via the GUI.
